(19) United States
(12) Patent Application Publication
(10) Pub. No.: US 2024/0358608 A1
(43) Pub. Date: Oct. 31, 2024

(54) POWDER FILLING TECHNOLOGY FOR AEROSOL CANISTERS

(71) Applicant: Tri-Pac, Inc., South Bend, IN (US)

(72) Inventors: Paras Shah, Granger, IN (US); Vikram Shah, Granger, IN (US); Pushker Sona, Granger, IN (US)

(21) Appl. No.: 18/638,935

(22) Filed: Apr. 18, 2024

Related U.S. Application Data
(60) Provisional application No. 63/498,612, filed on Apr. 27, 2023.

(51) Int. Cl. A61K 8/04; A61Q 5/02; A61Q 17/00; B65B 31/00

(52) U.S. Cl. CPC A61K 8/046; A61Q 5/02; A61Q 17/00; B65B 31/003; A61K 2800/87

(57) ABSTRACT

The present invention relates to filling technology for aerosol canisters. The present invention comprises in part a process for filling aerosol canisters by the steps of adding powder and liquid to the canister in separate phases before propellant.

### CROSS-REFERENCE TO RELATED APPLICATIONS

The present application claims priority U.S. provisional application Ser. No. 63/498,612, filed Apr. 27, 2023, herein incorporated by reference in its entirety.

### BACKGROUND

This invention relates to filling technology for aerosol canisters, and related processes and products.

Aerosol filling technology currently fills an aerosol canister in two stages. The first stage includes a liquid or slurry that may have dissolved or suspended powders or solids added to the canister. The second stage is a propellant stage, to add propellant such as compressed gas under pressure to the canister. The propellant stage in particular may include multiple filling stations.

Slurries and other liquid/solid combinations are often difficult to fill and require special filling equipment. Also, the powder premixed in the slurry can often drop out of solution, resulting in ineffective and low delivery that can result in a poor finished product. Also, in current processes, the liquid may be saturated by the solid and lose its character or potency. To prepare an acceptable slurry, a mill may be required, requiring additional processing time and capital expenditures up to $400,000 or more. A poorly mixed slurry may result in different powder particle sizes that may block the actuator and result in defective aerosol canisters.

### SUMMARY OF THE INVENTION

The present invention relates to filling technology for aerosol canisters. The present invention comprises in part a process for filling aerosol canisters by the steps of adding powder and liquid to the canister in separate phases before propellant.

In an embodiment, the process comprises filling an aerosol canister and preparing a finished aerosol product, comprising the steps of adding a powder to the canister and then adding a liquid to the canister; or adding a liquid to the canister and then adding a powder to the canister; and then completing filling (e.g. “propelling”) the canister with a propellant (including e.g. compressed gas). The process and method comprises powder filling equipment for filling the powder phase, a finished aerosol canister product, and an improved aerosol composition.

### BRIEF DESCRIPTION OF THE DRAWINGS

FIG. 1 is a side view of an actuator for use in one embodiment.

FIG. 2 is a cross-sectional side view of a valve system for use in one embodiment.

FIG. 3A is a side view of an actuator for use in one embodiment; FIG. 3B is a cross-sectional view of the actuator taken along line A-A in FIG. 3A; FIG. 3C is an enlarged view of the actuator taken from circle X is FIG. 3B; and FIG. 3D is a table of the insert orifice measurements.

FIG. 4A is a cross-sectional view of a valve and valve components for use in one embodiment; FIG. 4B is an exploded view of the valve components in one embodiment; and FIG. 4C is a table listing the valve components.

FIG. 5A is a side view of a canister for use in one embodiment; and FIG. 5B is an enlarged view of section A from FIG. 5A of the canister, according to one embodiment.

### DETAILED DESCRIPTION

The below discussion and definitions are intended to guide understanding but are not intended to be limiting with regard to other disclosures in this application. Throughout this application, references to percentage (%) of compositions of the present invention refers to the % by weight of a given substance to the total weight of the composition being discussed, also signified by “w/w” or “wt/wt”, unless stated otherwise.

Generally speaking, the present invention is directed to filling an aerosol canister without having to form a liquid-powder slurry prior to filling. The process of filling an aerosol canister without having to form a liquid-powder slurry prior to filling comprises at least 3 components (powder, liquid, and propellant) to be filled in three (3) distinct phases in an aerosol canister. In an embodiment, the present invention is directed to replacing the conventional, single-step process of filling a combination of powder and liquid components into a canister, with the present two-phase process of filling powder first and liquid after, or liquid first and powder after.

In an embodiment, a process of the present invention comprises, consists essentially of, or consists of at least the two-phase process described herein. In an embodiment, a product or aerosol composition of this invention comprises, consists essentially of, or consists of powder, liquid, and propellant of this invention.

In an embodiment of a process of this invention, Phase 1 is (i.e. comprises, consists essentially of, or consists of) the powder phase, during which powder is added to the aerosol canister (for instance free-flowing or non-free-flowing or amorphous or crystals or any form of solid); Phase 2 is (i.e. comprises, consists essentially of, or consists of) the liquid phase, during which liquid is added to the aerosol canister (where the liquid may be water based/solvent based, and may be a typical liquid and/or for instance an emulsion or paste); and Phase 3 is (i.e. comprises, consists essentially of, or consists of) the propellant phase, during which propellant (e.g. a propellant such as a compressed gas) is added to the aerosol canister to complete the filling of the canister and prepare a filled aerosol canister and a final aerosol product. The Phase 3 propellant phase occurs after Phase 2, and is not interchangeable with Phase 1 or Phase 2.

In an embodiment, Phase 1 is (i.e. comprises, consists essentially of, or consists of) the liquid phase, during which liquid is added to the aerosol canister (where the liquid may be water based/solvent based, and may be a typical liquid and/or for instance an emulsion or paste); Phase 2 is (i.e. comprises, consists essentially of, or consists of) the powder phase, during which powder is added to the aerosol canister (for instance free-flowing or non-free-flowing or amorphous or crystals or any form of solid); and Phase 3 is (i.e. comprises, consists essentially of, or consists of) the propellant phase, during which propellant (e.g. a propellant such as a compressed gas) is added to the aerosol canister to complete the filling of the canister and prepare a filled aerosol canister and a final aerosol product. The Phase 3 propellant phase occurs after Phase 2, and is not interchangeable with Phase 1 or Phase 2.

In an embodiment, powder filling technology, liquid filling technology, and/or propellant filling technology are/is conventional. In an embodiment, the present invention utilizes all three technologies (powder, liquid, and propellant) to create an aerosol spray with a heavy powder content. The conventional technology uses a slurry phase (powder and liquid premixed requiring continuous agitation and/or circulation) along with a propellant phase addition. The current invention utilizes powder, liquid, and propellant as separate phases which allows the powder to remain in powder format without any contact with liquid; in an embodiment said contact with liquid may reduce potency, efficiency, size, shape, and adsorbing or absorbing capabilities of the powder. The three phase filling technology also allows for ensuring all three phases of the correct proportions are added to the canister. In the event of the older technology using slurry, the powders (either suspended or solubilized) have a tendency to “fall out” resulting in incorrect, improper and/or inconsistent dosing amounts of the powder in the final canister.

In the present invention, in an embodiment and without being bound by theory, powder and liquid are not introduced to each other until the point of filling (i.e. added to the canister), preventing or minimizing particle agglomeration and preventing decreases in product efficacy. Without being bound by theory, by reducing the interaction between the liquid and powder until filling, the powder is able to come into contact with the propellant more quickly and maintain its potency and useful structure.

The present invention provides several advantages over conventional aerosol filling technology. Significant advantages include improved finished product performance and improved product delivery, improved ease of handling, labor savings due to lower batching and compounding times and processes, lower capital expenses. Also, significant advantages include reduced batch time, reduced need for circulation, reduced risk of poor product, and improved effectiveness of the powder. Also, while a powder filler according to the present invention may require a capital expenditure of up to $200,000, such cost is roughly half the cost of powder mills that are needed for some current aerosol fillers, which may cost up to $400,000.

In an embodiment, a process of the present invention may be used in any aerosol canister filling process that requires or may require a powder load for instance in the liquid stage of the product, including for instance an aerosol canister that conventionally would be filled after dissolving or suspending the powder in a liquid for instance to form a slurry.

In an embodiment the powder added during the powder phase (Phase 1 or 2) is from about 0.1% to about 60% w/w of the liquid added during the liquid phase (Phase 1 or 2). In an embodiment the liquid added during the liquid phase (Phase 1 or 2) is from about 40% to about 99.9% w/w of the powder added during the powder phase (Phase 1 or 2).

In an embodiment of this invention, including a product or process, powder is from 1-25%, liquid is from 1-50%, and propellant is from 1-90%.

A “powder” e.g. as used in a Powder Phase according to the present invention, in an embodiment, comprises benzocaine, hydrocortisone, lidocaine, polyvinylpyrrolidone-iodine, colloidal oatmeal, aluminum chlorohydrate, aluminum chloride, aloe vera, pramoxide hydrochloride, tapioca starch, silica, rice starch, titanium oxide, zinc oxide, tolnaftate, miconazole nitrate, menthol, camphor, salicylic acid, cetylpyridinium chloride, budenoside, naloxone hydrochloride, phenylephrine, and/or any other active ingredient(s) or functional ingredient(s) or other ingredients that may exist in a powder or solid format. In an embodiment, a powder used in a Powder Phase of this invention comprises, consists of, or consists essentially of rice starch, cetrimonium chloride, kaolin, and distearyldimonium chloride. In an embodiment, a powder used in a Powder Phase of this invention comprises, consists of, or consists essentially of a mixture of corn starch, rice starch, and/or tapioca starch, benzothonium chloride, calamine powder, kaolin, sodium bicarbonate, and magnesium stearate. In an embodiment, the powder in the Powder Phase is about 5-15% w/w of the canister components, including e.g. 7-12% w/w, 7-10% w/w, or 8-12% w/w.

A “liquid” e.g. as used in a Liquid Phase according to the present invention in an embodiment of a product or process of the present invention can be any flowing liquid (i.e. that can be considered a liquid). For instance, in an embodiment, a liquid of this invention may be one or more of a solubilizing agent, suspending agent, and/or carrier. For instance, in an embodiment of this invention, a liquid may comprise alcohol(s), silicone(s), humectant(s), emollient(s), water, UVA/UVB agent(s), vegetable Oil(s), synthetic oil(s), and/or other flowing liquid(s) as discussed above. In an embodiment, a liquid used in a Liquid Phase of this invention comprises, consists essentially of, or consists of a mixture of ethanol, fragrance, extract(s), and silicone(s). In an embodiment, a liquid used in a Liquid Phase of this invention comprises, consists of, or consists essentially of a mixture of alcohol, silicone, sorbitan oleate, bisabolol, isopropylmyristate, fragrance, and essential oil(s). In an embodiment, the liquid in the Liquid Phase is about 2-20% w/w of the canister components, including e.g. 3-12% w/w, 3-6% w/w, or 8-12% w/w.

A “propellant” used in a Propellant Phase according to the present invention may be any propellant including e.g. a propelling chemical, e.g. a compressed gas, that may be filled into a canister and produce an aerosol. In an embodiment of a product or process of the present invention, propellant comprises any propellant or propelling chemical including but not limited to, in whole or partially, isobutane, butane, n-butane, propane, HFC 152a, 245fa, 1234ze, HFO-1234ze, 1233zd, HFO-1233zd, HFA134 a, HFA 134A/p, pentane, isopentane, nitrogen, compressed gas(es) such as compressed air, carbon dioxide, nitrogen dioxide, nitrous oxide, argon, and/or dimethyl ether, and/or any blend of any of the above or other propellants for instance, propellant mixture(s). A Propellant Phase of the present invention may include conventionally known or other processes for adding propellant to an aerosol canister, including for instance inline propellant blending. In an embodiment, a propellant used in a Propellant Phase of this invention comprises, consists of, or consists essentially of a mixture of propellants such as HFC 152a, propane, and isobutane. In an embodiment, the propellant in the Propellant Phase is about 75-90% w/w of the canister components, including e.g. 1-80%, 50-90%, 80-90%, or 75-85% w/w.

An “aerosol canister” according to the present invention refers to a canister that will have or has powder, liquid, and propellant under pressure to be dispensed as an aerosol. In an embodiment, the canister is a container that may be used to hold and dispense an aerosol. In an embodiment, an aerosol canister is a self-propelled canister. In an embodiment, an aerosol canister according to the present invention is through-the-valve, metered valve, under the cup, or is prepared with micro-filling technology.

In an embodiment, an aerosol canister product of the present invention comprises an aerosol canister of this invention having been filled with Phases 1, 2, and 3. Said aerosol product may include aerosol formulations inclusive of for instance topical, oral, and/or hair products, for instance for personal care; medical devices; over the counter; or any other aerosol products. A canister may be sized for a desired use, including for instance hand-held use.

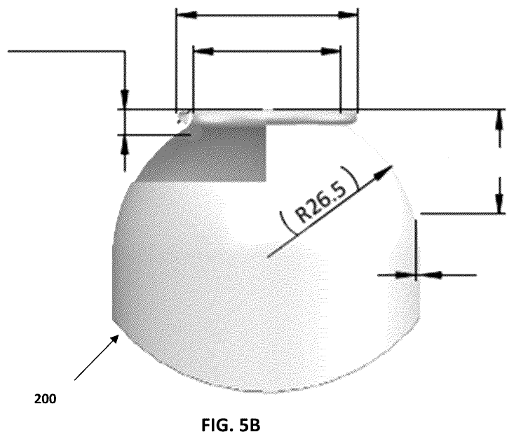

An example of a canister of the present invention is shown in FIG. 5. Dimensions are presented in millimeters, unless otherwise stated. The canister is prepared with an aluminum alloy having 95% or more aluminum (e.g. 99.7%). The canister is lined with a phenolic epoxy. The canister provides a minimum of metal exposure (e.g. 50 mA maximum). The opening is 1″ outside. The canister has a pressure rating of DOT 2P, overflow of 259 mL, and unfilled/empty container weight.

In an embodiment, powder filling technology (e.g. equipment) according to this invention is or may be commercially available equipment, in an embodiment that was customized to include special filling nozzles that place powder directly into a canister such as a 1 inch canister opening without any powder deposit such as visible powder deposit on the chime of the canister. Additionally, in an embodiment, special coatings and augers were or may be customized to create accurate dosing for free-flowing powders and non-free flowing powders so the equipment can handle all powder technologies despite their flow or coagulating characteristics. In an embodiment, the equipment was or may be customized to make the unit intrinsically safe e.g. for operations around flammable powders and/or electrostatically charged powders.

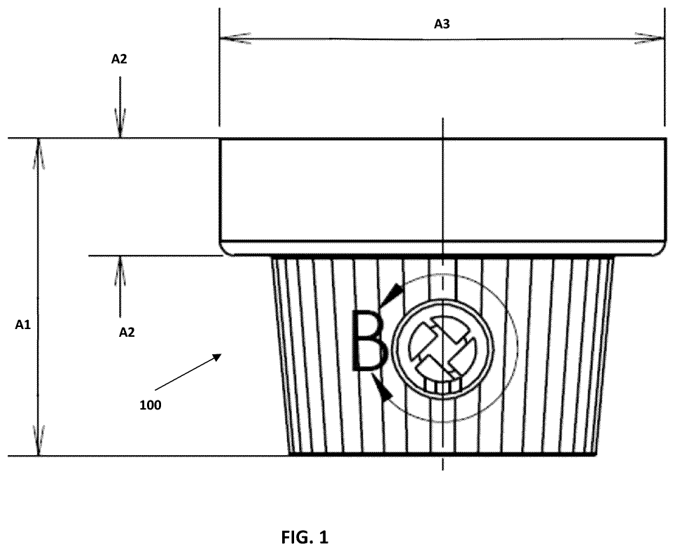

FIG. 1 is a drawing of an actuator 100 for use in one embodiment, for instance for preparing a dry shampoo powder spray as described in Example 1. The actuator 100 includes a height A1, a top portion thickness A2, and a width A3. A1 is between about 15 and 17 mm, A2 is between about 5 and 7 mm, and A3 is between about 21 and 24 mm. Dimensions shown may be varied to accommodate equipment of different sizes. Dimensions shown may not be to scale. Drawing scale is in millimeters (mm). Actuator is nonmechanical break up actuator. Specifications: basic actuator made of polypropylene, insert (Blk. 0.035″ orifice×0.036″ land length) is acetal. (Summit Europe, The Netherlands).

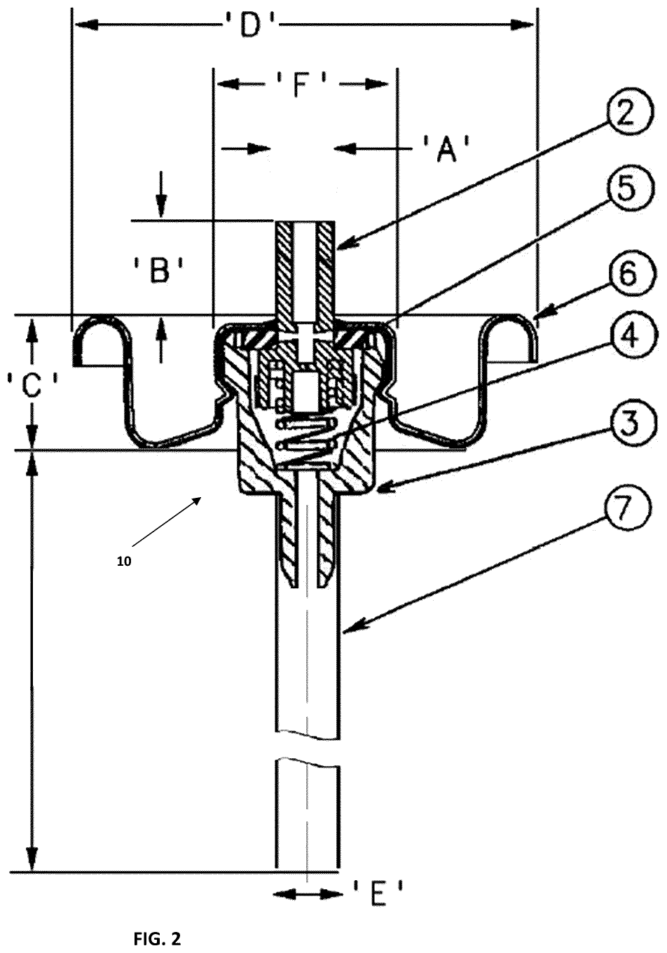

FIG. 2 is a cross sectional view of a valve system 10 for use in one embodiment, for instance for preparing a dry shampoo powder spray as described in Example 1. Dimensions shown may be varied to accommodate equipment of different sizes. Dimensions shown are not to scale. The valve system comprises a stem 2 (1×0.025×0.349 Nat, material: acetal), a body 3 (0.062 RE 0.031 VT, material: nylon), a spring 4 (0.023, material: 302 stainless steel), a gasket 5 (0.050 BUNA Summit SV-77/92; material BUNA), a cup 6 (M.CUP EPT/LAMB CON RlNek, material: TINPLATE/POLYPRO), and a tube 7 all operable to function and maintain the valve system 10 (standard cut, material: LD polyethylene). Dimension ‘A’ refers to stem outer diameter, measurement about 4.03 mm, tolerance±0.04 mm. Dimension ‘B’ refers to stem to curl of cup, measurement about 6.48 mm, tolerance±0.38 mm. Dimension ‘C’ refers to cup height, measurement about 9.40 mm, ±0.13 mm. Dimension ‘D’ refers to cup width, measurement about 32.52 mm, tolerance±0.08 mm. Dimension ‘E’ refers to dip tube outer diameter, measurement about 4.32 mm, tolerance±0.05 mm. Dimension ‘F’ refers to pedestal diameter, measurement about 12.64 mm, tolerance±0.13 mm. FBOC length for dip-tube with tube straight: about 160 mm. For can size 45×178. (Summit Europe, The Netherlands).

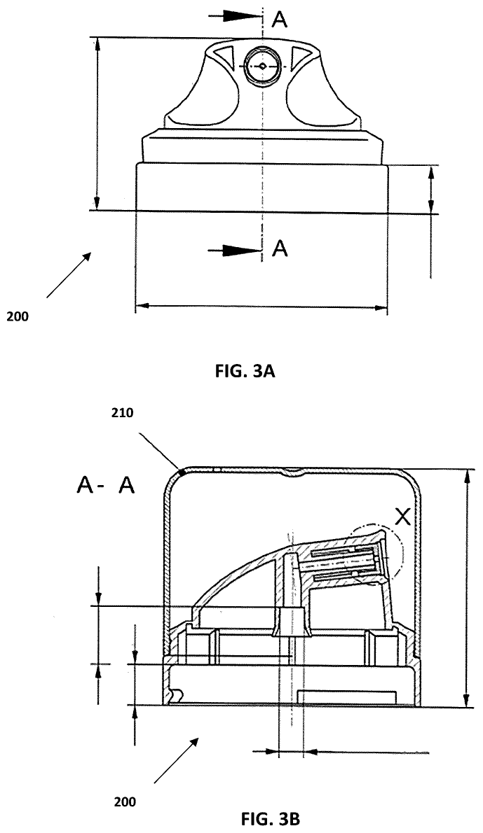

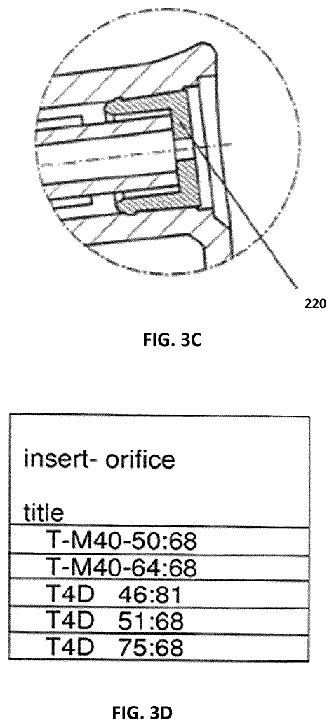

FIG. 3A is a side view an actuator 200 for use in one embodiment, for instance for an antiperspirant product. FIG. 3B shows the actuator 200 with the overcap 210. FIG. 3C shows the insert in the orifice of the actuator 200. Dimensions shown may be varied to accommodate equipment of different sizes. Dimensions shown may not be to scale. (Lindal Dispenser GmbH, Schonberg, Germany). FIG. 3D is a table of the insert orifice measurements.

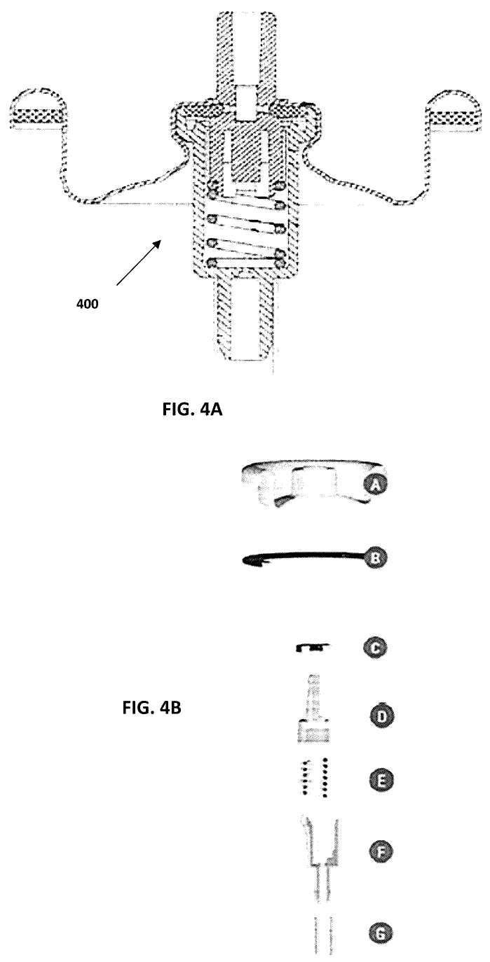

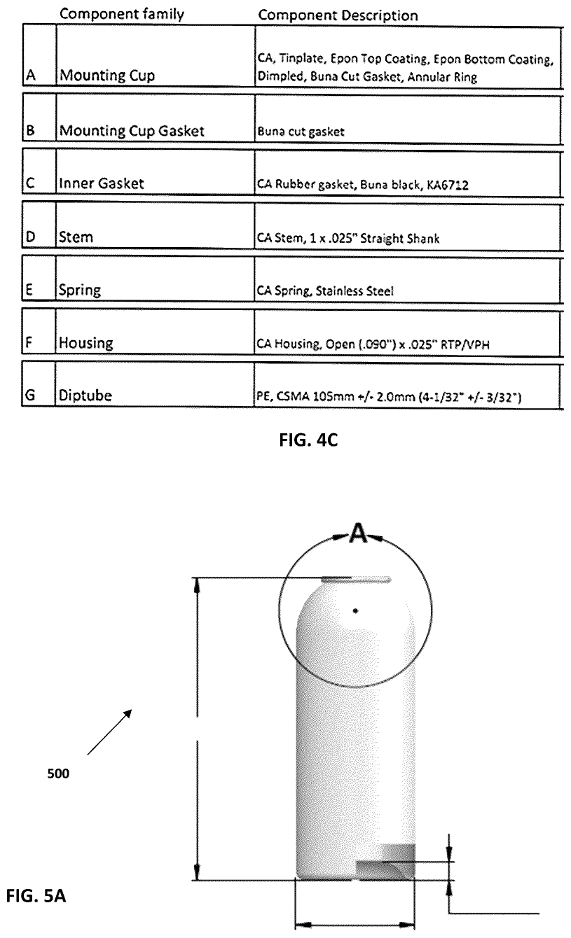

FIG. 4A is a cross-sectional view of a valve 400 and valve components for use in one embodiment. Dimensions shown may be varied to accommodate equipment of different sizes. Dimensions shown may not be to scale. FIG. 4B is an exploded view of the valve assembly 400 including the valve components A (mounting cup), B (mounting cup gasket), C (inner gasket), D (stem), E (spring), F (housing), G (diptube). (Lindal Group, Lindal North America, Columbus, Indiana). FIG. 4C is a table listing the valve components and component description.

FIG. 5A is a side view a canister 500 for use in one embodiment. FIG. 5B is an enlarged view of A from FIG. 5A, showing the radius of curvature at about 26.5. Dimensions shown may be varied to accommodate equipment of different sizes. Dimensions shown may not be to scale. Units shown are in millimeters (mm), unless otherwise stated. Canister description: Aluminum Alloy (99.7% A1, 1070), white exterior, round shoulder, epoxyphenolic inside liner, maximum metal exposure: 50.0 mA, 1″ outside opening, pressure rating: DOT 2P, weight: 28 g (ref), overflow: 259 mL (ref), machine curl.

The present invention may be further understood in connection with the following Examples and embodiments. The following non-limiting Examples and also embodiments described throughout this application are provided to illustrate the invention.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Dry Shampoo Powder Spray

A dry shampoo powder spray is prepared according to the following 3 Phases:

| Phase 1 | Phase 2 | Phase 3 |
|---|---|---|
| Mixture of Rice Starch, | Mixture of Ethanol, | Propellant or mixture of |
| Cetrimonium Chloride, | fragrance, extracts, silicones | propellants-HFC152A, |
| Kaolin, Distearyldimonium | | Isobutane, Propane |
| Chloride | | |
| 7-10% w/w | 3-6% w/w | 80-90% w/w |

### Example 2: Anti-Chafing Spray

An anti-chafing spray is prepared according to the following 3 Phases:

| Phase 1-Powder blend | Phase 2-Liquid blend | Phase 3-Propellant blend |
|---|---|---|
| Mixture of Corn/Rice/ | Mixture of alcohol, silicone, | Mixture of propellants-HFC |
| Tapioca Starch, | sorbitan oleate, bisabolol, | 152a, propane, isobutane |
| Benzothonium Chloride, | isopropylmyristate, fragrance, | |
| Calamine Powder, Kaolin, | essential oils | |
| Sodium Bicarbonate, | | |
| Magnesium Stearate | | |
| 8-12% w/w | 8-12% w/w | 75-85% w/w |

The powder filling equipment is a commercially available equipment that was customized to include special filling nozzles that place powder directly into the 1 inch canister opening without any powder deposit on the chime of the canister. Additionally, special coatings and augers were customized to create accurate dosing for free-flowing powders and non-free flowing powders so the equipment can handle all powder technologies despite their flow or coagulating characteristics. Additionally, the equipment was customized to make the unit intrinsically safe for operations around flammable powders and/or electrostatically charged powders.

The use of the terms “a,” “an,” “the,” and similar referents in the context of describing the present invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±10%; in other embodiments, the values may range in value above or below the stated value in a range of approximately ±5%; in other embodiments, the values may range in value above or below the stated value in a range of approximately ±2%; in other embodiments, the values may range in value above or below the stated value in a range of approximately ±1%. The preceding ranges are intended to be made clear by context, and no further limitation is implied. All method steps described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise stated. No language in the specification should be construed as indicating any non-claimed element as essential to the practice of the invention.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, mechanical, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

While in the foregoing specification the present invention has been described in relation to certain embodiments thereof, and many details have been put forth for the purposes of illustration, it will be apparent to those skilled in the art that the invention is susceptible to additional embodiments and that certain of the details described herein can be varied considerably without departing from the basic principles of the invention.

The present invention may be embodied in other specific forms without departing from the spirit or essential attributes thereof, and, accordingly, reference should be made to the appended claims, rather than to the foregoing specification, as indicating the scope of the invention.

1. The process of filling an aerosol canister without having to form a liquid-powder slurry prior to filling an aerosol canister, comprising a first phase, a second phase, and a third phase; the first phase comprising adding powder to an aerosol canister; the second phase comprising adding liquid to the aerosol canister, wherein the powder remains in powder format without any contact with liquid; and the third phase comprises adding propellant to the aerosol canister to complete the filling of the canister and the powder is able to come into contact with the propellant more quickly and maintain its potency form a final aerosol product with a heavy powder content.

2. The process of claim 1, wherein the first phase comprises free-flowing or non-free-flowing or amorphous or crystals or any form of solid; the second phase comprises the liquid as a water based/solvent based or a typical liquid and/or for instance an emulsion or paste; and the third phase comprises propellant as a compressed gas; and the third phase occurs after the second phase and the second phase occurs after the first phase.

3. The process of claim 2, wherein the powder and liquid are not introduced to each other until the point of adding to the canister and preventing particle agglomeration; and reducing the interaction between the liquid and powder until filling, the powder is able to come into contact with the propellant.

4. The process of claim 3, wherein after step c, the filled aerosol canister is a finished aerosol product, and wherein the finished aerosol product is a topical product such as an anti-chafing spray, an oral product, a hair product such as a dry shampoo spray, a medical device, an Antiperspirant spray and/or an over-the counter product spray.

5. The process of claim 4, wherein the first phase comprises a mixture of Rice Starch, Cetrimonium Chloride, Kaolin, Distearyldimonium Chloride between about 7 and about 10% w/w; the second phase comprises a mixture of Ethanol, fragrance, extracts, silicones between about 3% w/w and about 6% w/w; and the third phase comprises a propellant or mixture of propellants selected from the group consisting of HFC152A, Isobutane, and Propane between about 80% w/w and about 90% w/w.

6. The process of claim 4, wherein the first phase comprises a powder blend including a mixture of Corn/Rice/Tapioca Starch, Benzothonium Chloride, Calamine Powder, Kaolin, Sodium Bicarbonate, Magnesium Stearate between about 8% w/w and about 12% w/w; the second phase comprises a liquid blend including a mixture of alcohol, silicone, sorbitan oleate, bisabolol, isopropylmyristate, fragrance, essential oils between about 8% w/w and about 12% w/w; and the third phase comprises a propellant blend including a mixture of propellants including HFC 152a, propane, and isobutane between about 75% w/w and about 85% w/w.

7. The process of claim 6, further comprising powder filling equipment include special filling nozzles that place powder directly into the a 1-inch canister opening without any powder deposit on a chime of the canister.

8. An aerosol product comprising powder, liquid, and propellant such as compressed gas prepared according to the process of claim 4.

9. An aerosol composition comprising the powder, liquid, and propellant of claim 4.

10. A process for filling an aerosol canister comprising the steps of:
a. adding a powder or a liquid to the aerosol canister, and then
b. adding a liquid to the powder in the canister or adding a powder to the liquid in the canister; and
c. adding a propellant such as compressed gas to the aerosol canister to complete filling of the canister.

11. The process of claim 10, wherein after step c, the filled aerosol canister is a finished aerosol product.

12. The process of claim 11, wherein the finished aerosol product is a topical product such as an anti-chafing spray, an oral product, a hair product such as a dry shampoo, a medical device, an Antiperspirant and/or an over-the counter product.

13. The process of claim 12, wherein the dry shampoo powder spray is prepared according to a first phase, a second phase, and a third phase, wherein the first phase comprises a mixture of Rice Starch, Cetrimonium Chloride, Kaolin, Distearyldimonium Chloride between about 7 and about 10% w/w; the second phase comprises a mixture of Ethanol, fragrance, extracts, silicones between about 3% w/w and about 6% w/w; and the third phase comprises a propellant or mixture of propellants selected from the group consisting of HFC152A, Isobutane, and Propane between about 80% w/w and about 90% w/w.

14. The process of claim 13, wherein the anti-chafing Spray is prepared according to a first phase, a second phase, and a third phase; wherein the first phase comprises a powder blend including a mixture of Corn/Rice/Tapioca Starch, Benzothonium Chloride, Calamine Powder, Kaolin, Sodium Bicarbonate, Magnesium Stearate between about 8% w/w and about 12% w/w; the second phase comprises a liquid blend including a mixture of alcohol, silicone, sorbitan oleate, bisabolol, isopropylmyristate, fragrance, essential oils between about 8% w/w and about 12% w/w; and the third phase comprises a propellant blend including a mixture of propellants including HFC 152a, propane, and isobutane between about 75% w/w and about 85% w/w.

15. The process of claim 13 further comprising powder filling equipment include special filling nozzles that place powder directly into the a 1-inch canister opening without any powder deposit on a chime of the canister.

16. An aerosol product comprising powder, liquid, and propellant such as compressed gas prepared according to the process of claim 12.

17. An aerosol composition comprising the powder, liquid, and propellant of claim 12.
